(19) United States
(12) Patent Application Publication
(10) Pub. No.: US 2017/0347902 A1
(43) Pub. Date: Dec. 7, 2017

(54) OPTICAL VITAL SIGNS SENSOR

(71) Applicant: KONINKLIJKE PHILIPS N.V., EINDHOVEN (NL)

(72) Inventors: Edgar Martinus VAN GOOL, Veghel (NL); Elvira Johanna Maria PAULUSSEN, Reppel-Bocholt (BE)

(21) Appl. No.: 15/536,363

(22) PCT Filed: Dec. 2, 2015
(86) PCT No.: PCT/EP2015/078262

§ 371 Date: Jun. 15, 2017

(30) Foreign Application Priority Data
Dec. 16, 2014 (EP) 14198293.4

(51) Int. Cl. A61B 5/024

(52) U.S. Cl. CPC A61B 5/02427; A61B 5/02438; A61B 2562/0238; A61B 2562/043; A61B 2562/0242

(57) ABSTRACT

An optical vital signs sensor comprises a light source (110) having alight unit (111, 112) generating light which is directed towards a skin (1000) of a user, at least one photo detector unit (120) having a plurality of photo diodes (121-12n) detecting light from the skin (1000), and an adjusting unit (140, 150) configured to adjust an effective distance which the light travels between the light unit (111, 112) and the photo diode (12-12n).

### FIELD OF THE INVENTION

The invention relates to an optical vital signs sensor and a method of operating an optical vital signs sensor.

### BACKGROUND OF THE INVENTION

Optical heart rate sensors are well known to monitor or detect vital signs like a heart rate of a user. Such a heart rate sensor can be based on a photoplethysmographic PPG sensor and can be used to acquire a volumetric organ measurement. By means of pulse oximeters, changes in light absorption of a human skin is detected and based on these measurements a heart rate or other vital signs of a user can be determined. The PPG sensors comprise a light source like a light emitting diode LED which is emitting light into the skin of a user. The emitted light is scattered in the skin and is at least partially absorbed by the blood. Part of the light exits the skin and can be captured by a photodiode. The amount of light that is captured by the photo diode can be an indication of the blood volume inside the skin of a user. A PPG sensor can monitor the perfusion of blood in the dermis and subcutaneous tissue of the skin through an absorption measurement at a specific wave length. If the blood volume is changed due to the pulsating heart, the scattered light coming back from the skin of the user is also changing. Therefore, by monitoring the detected light signal by means of the photodiode, a pulse of a user in his skin and thus the heart rate can be determined.

US 2014/0275852 A1 discloses a wearable fitness monitoring device including a motion sensor and a PPG sensor.

US 2013/0261415 A1 discloses an optical vital signs sensor which operates in a transmissive mode and comprises several light emitting diodes and several photo diodes. The sensor comprises a processing unit configured to dynamically configure an operational geometry of the sensor by controlling the intensity of one or more of the light emitting diodes and the gain of at least one of the photo detectors.

US 2010/0081901 A1 discloses an optical vital signs sensor having optical light emitting elements and light detecting elements. The light detecting elements can be adaptive light detecting elements to selectively detect light of certain wavelengths or from certain regions of the tissue of the user.

WO 2014/184447 A1 relates to a portable pulse measuring device having several light emitting sources and a light detector for detecting the intensity of the reflective light. The light emitting sources are arranged in an asymmetrical configuration in relation to the light detectors.

US 2010/0113948 A discloses a PPG sensor arranged behind the ear of a user.

FIG. 1 shows a basic representation of an operational principle of an optical heart rate sensor. In FIG. 1, a heart rate sensor is arranged on an arm of a user. The heart rate sensor 100 comprises a light source 110 and a photo detector 120 arranged at a fixed distance. The light source 110 emits light onto or in the skin 1000 of a user. Some of the light is reflected and the reflected light can be detected by the photo detector 120. The optical heart rate sensor can be embodied as a PPG sensor. Such a PPG sensor is however not easily adaptable to different operating scenarios.

### SUMMARY OF THE INVENTION

It is an object of the invention to provide an optical vital signs sensor which is able to efficiently detect vital signs of a user during different operating conditions.

According to an aspect of the invention, an optical vital signs sensor is provided which comprises a light source having at least one light unit each configured to generate light. The light of the light source is directed towards the skin of the user. The optical vital signs sensor furthermore comprises at least one photo detector unit having at least one photo diode configured to detect light which is indicative of a reflection of the light from the at least one light unit in or from a skin of a user. The sensor furthermore comprises an adjusting unit (which can be mechanical or optical) configured to mechanically adjust a position of one of the at least one light unit relative to a position of one of the at least one photo diode thereby adjusting the distance between an active light unit and an active photo diode.

According to a further aspect of the invention, the sensor furthermore comprises a control unit configured to control an operation of the adjusting unit based on a signal quality of an output signal of the photo detector analyzed by a signal analyzing unit in the control unit thereby further adjusting a distance between an active light unit and an active photo diode.

According to an aspect of the invention, an optical vital signs sensor comprising a light source having at least one light unit configured to generate light is provided. The light of the light source is directed towards a skin of a user. The optical vital signs sensor further comprises at least one photo detector unit having a plurality of photo diodes configured to detect light which is indicative of a transmission or reflection of the light from the at least one light unit in or from the skin of the user. The adjusting unit can comprise or be implemented as a control unit configured to control an operation of the light source and/or the photo detector unit based on a signal quality of an output of the photo detector as analyzed by a signal analyzing unit in the control unit. At least one light unit from among the at least one light unit and/or at least one photo diode from among the plurality of photo diodes is selected and activated thereby adjusting a distance between an active light unit and an active photo diode or adjusting a position where the light from the at least one light source enters the skin of the user.

According to a further aspect of the invention, the signal quality is analyzed by the signal analyzing unit regarding at least one of a signal-to-noise ratio, an amplitude of the DC component of the output signal, an amplitude of the AC component of the output signal and a ratio between the AC and DC component of the output signal of the photo detector.

According to an aspect of the invention, a computer program product comprising a computer readable memory storing computer program code means for operating the optical vital signs sensor is provided.

While known optical vital signs sensors like heart rate sensors have a predetermined and fixed distance between the light source and the photo detector, the aspects of the invention relate to an optical vital signs sensor which allows a variation of the distance between the light source and the photo detector. By providing a variation mechanism, the optical vital signs sensor according to an aspect of the invention allows an adaptation on different detecting situations like different users, different climatic conditions. It should be noted that a larger distance between the light source and the detector is advantageous as the AC portion of the output signal increases over the DC portion of the output signal. On the other hand, if the distance between the light source and the photo detector is too high, the signal-to-noise ratio will suffer. The signal-to-noise ratio as well as the battery life may suffer. The optical vital signs sensor according to an aspect of the invention allows an adaptation of the distance between the light source and the photo detector. This variation can be achieved by changing the distance mechanically between the light source and the photo detector. Alternatively, a plurality of light sources and/or photo detectors can be provided as a control unit can select at least one light source and at least one photo detector from among the plurality of light sources and/or photo detectors, which can be arranged at different positions in order to achieve the desired distance between the light source and the photo detector. Alternatively, a light guide can be provided between the light source and a photo diode placed above a skin of a user, wherein such a light source comprises several controllable outputs at different positions. The position of the output of the light out of the light guide with reference to the photo detector is varied. The controllable outputs can be implemented by a steerable optical reflection layer which can be a liquid crystal or an electrochromic layer.

It shall be understood that a preferred embodiment of the present invention can also be a combination of the dependent claims or above embodiments or aspects with respective independent claims.

These and other aspects of the invention will be apparent from and elucidated with reference to the embodiment(s) described hereinafter.

### BRIEF DESCRIPTION OF THE DRAWINGS

In the following drawings:

FIG. 1 shows a basic representation of an operational principle of an optical vital signs sensor,

FIG. 2 shows a schematic block diagram of an optical vital signs sensor according to an aspect of the invention,

FIGS. 3 to 7 each show a basic representation of an optical vital signs sensor according to aspects of the invention,

FIGS. 8 and 9 each show a representation of an optical vital signs sensor according to aspects of the invention, and

FIG. 10 shows a schematic representation of an optical vital signs sensor according to an aspect of the invention.

### DETAILED DESCRIPTION OF EMBODIMENTS

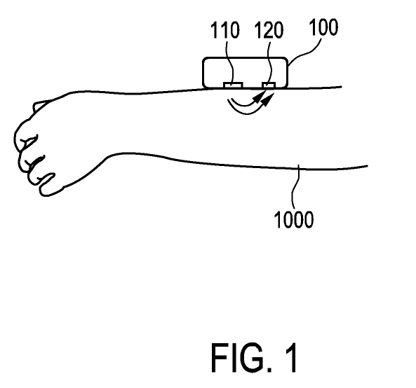

According to an aspect of the invention, an optical vital signs sensor is provided which is based on a photoplethysmograph PPG sensor. Such a PPG sensor is depicted in FIG. 1. A light source emits light onto or into a skin 1000 of a user and some of the light is reflected and the reflected light can be detected by a photo detector 120. The output of the photo detector can be analyzed to determine a heart rate or other vital signs of a user.

The PPG sensor or optical vital signs sensor according to an aspect of the invention can be implemented as a wrist device like a smart watch. The optical vital signs sensor according to an aspect of the invention can also be implemented as a device which is for example worn behind the ear of a user or at any other parts of the user as long as it is ensured that the optical vital signs sensor is placed directly onto the skin of a user.

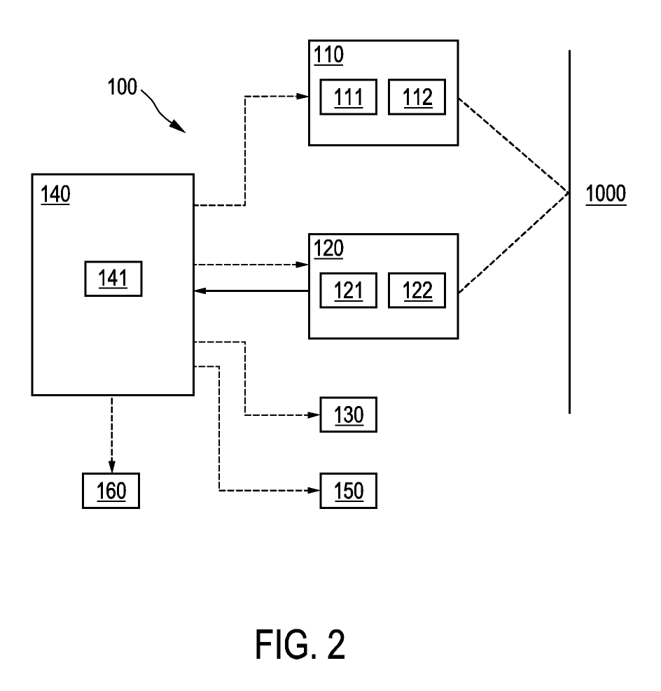

FIG. 2 shows a schematic block diagram of an optical vital signs sensor according to an aspect of the invention. The optical vital signs sensor 100 comprises a light source 110 and a photo detector 120. The light source 110 emits light onto or in the skin 1000 of a user at a certain position and the photo detector 120 is able to detect reflected or transmitted light. The light source may comprise several light units 111, 112 which can for example be implemented as light emitting diodes LED. The photo detector 120 may comprise several photo diodes 121, 122 which serve to detect light as reflected or transmitted from the skin 1000 of a user. The optical vital signs sensor 100 furthermore comprises a control unit 140 which can control the operation of the light source 110 and/or the photo detector 120. The control unit 140 can optionally receive the output of the photo detector 120. The control unit 140 comprises a signal analysis unit 141 which serves for analyzing the output signal of the photo detector 120.

Optionally, the optical vital signs sensor 100 can comprise an adjustable light guide 130 which serves to guide the light from the light source 110 onto the skin 1000 of a user. The light guide 130 can be arranged between the light source 110 and the skin 1000 of the user.

Furthermore, optionally, the optical vital signs sensor 100 can comprise an adjusting unit 150 which serves to adjust the distance between the light source 110 or one of the light units 111, 112 and the photo detector or the photo diodes 121, 122. The adjusting unit 150 can be controlled by the control unit 140 or can be adjusted by a user.

In addition, optionally, the optical vital signs sensor 100 comprises a display 160, by means of which an (adjustable) feedback can be provided. In particular, the feedback to the user can be implemented by a green or red light or by a transitioning from red to green or from red via yellow to green. This feedback serves to enable the user to adjust the optimum distance for the detector. In addition or alternatively, the feedback can be visual, audible or haptic or a combination of these. The feedback can be provided via the display 160 or via other feedback means like a loudspeaker, etc. The function of the feedback is to improve the adjustment of the sensor for optimum performance.

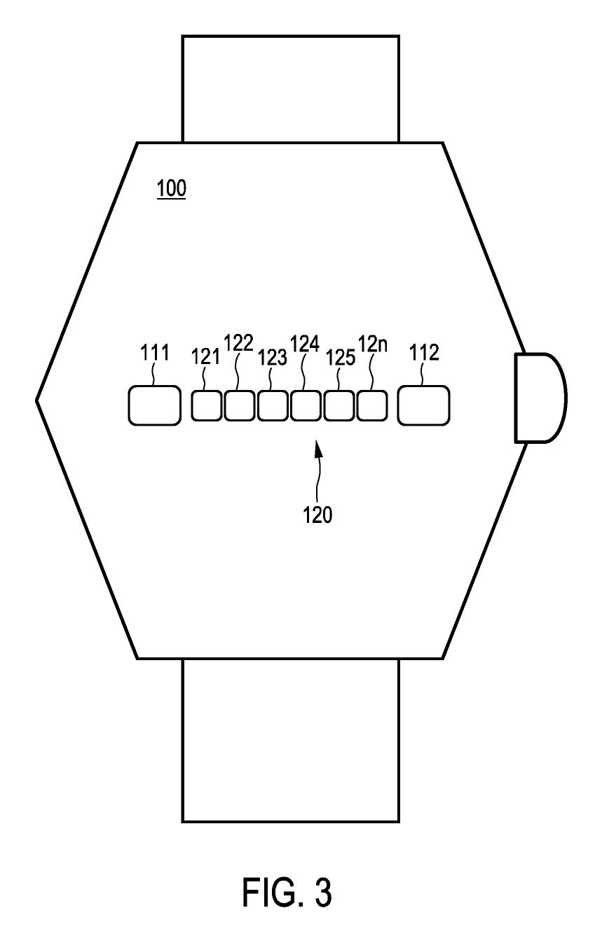

FIG. 3 shows a basic representation of an optical vital signs sensor according to aspects of the invention. According to this aspect of the invention, the optical vital signs sensor 100 is implemented as a wrist device like a smart watch. The optical vital signs sensor 100 comprises two light units 111, 112 which can be implemented as light emitting diodes LED. Between the first and second light unit 111, 112, the photo detector 120 with a plurality of photo diodes 121-12n each at a different position is provided. Optionally, a first and second light emitting diode 111, 112 and the photo diodes 121-12n are arranged in a line.

Optionally, all photo diodes 121-12n are activated and the signal analyzing unit 141 can receive the signals from all of the photo diodes 121-12n and can determine which signal has the best signal quality. The signal quality may be determined based on the signal to noise ratio or by the AC or DC amplitude or by the ratio between the AC and DC component. The control unit 140 may select one or a plurality of the photo diodes 121-12n which provides the best signal quality and may deactivate the other photo diodes 121-12n. The control unit 140 may activate all of the photo diodes either concurrently or subsequently in order to detect the signal quality of all of the photo diodes 121-2n and to decide which one has the best signal quality. The decision on the best signal quality may be determined on the signal-to-noise ratio, the best AC or DC amplitude or the best ratio between the AC and DC amplitude.

As each of the photo diodes 121-2n has a different position, the distance between the at least one light emitting diode 111, 112 and the selected photo diode 121-12n is changed. The variation of the distance between the active light emitting diode 111, 112 and the active photo diode 121-12n is selected by the control unit 140 for example based on the results of the signal analysis unit 141. According to this aspect of the invention, there is no mechanical change between the positions of the photo diode and the light emitting diode. In contrast, at least one of the photo diodes 121-12n is selected. Accordingly, redundant photo detectors can be provided. These can be used with three photo diodes or with pairs of three photo diodes and three light emitting diodes. The pair with the best signal or best signal quality can be selected in order to exclude an influence of black spots on the skin, namely spots where no signal is detected.

If the photo diodes are used concurrently, they can enhance or debilitate the signal. If the signal is to be enhanced, multiple photo diodes can be used simultaneously.

Optionally, the wave length of the first light emitting diode 111 may be different to the wavelength of the second light emitting diode. Although in FIG. 3 only two light emitting diodes are shown, it should be noted that the number of light emitting diodes may also be a larger one.

According to this aspect of the invention, the control unit 140 may select the first or the second light emitting diode 111, 112 or may select both of them simultaneously. However, a selection of one of the light emitting diodes is preferred. In particular, the light emitting diode which is resulting in the best signal quality at the photo diodes is selected and the output of the light emitting diodes can be increased by increasing its current input. This is advantageous as the robustness of the system is increased. The robustness is in particular important in view of a motion between the skin of the user and the sensor.

The selection of the light emitting diode which is resulting in the best output signal of the photo diode is advantageous in view of the power consumption of the sensor as the power consumption of one light emitting diode will be less than the power consumption of a multiple of light emitting diodes.

The control unit 140 is adapted to activate or deactivate the light emitting diodes 111, 112 and the photo diodes 121-12n to reduce the power consumption of the optical vital signs sensor.

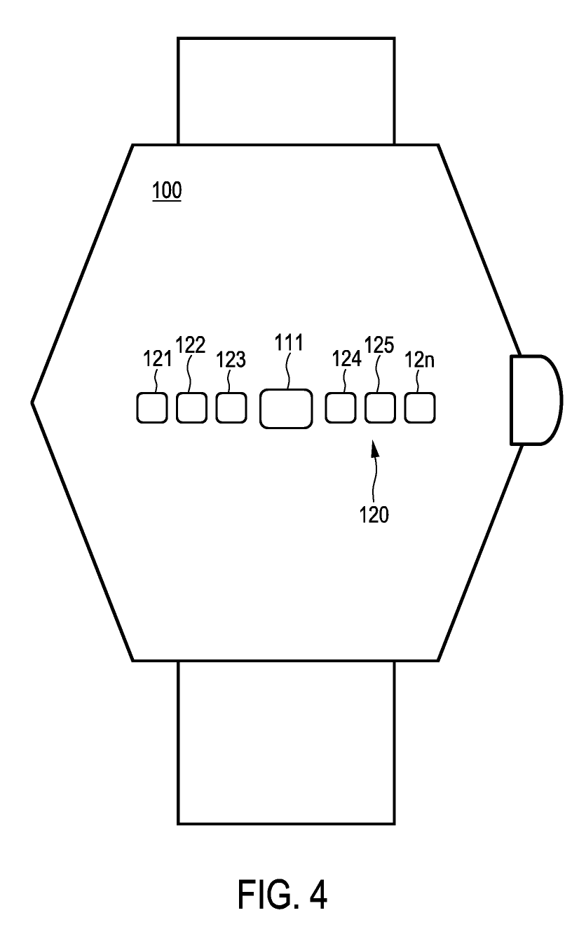

FIG. 4 shows a basic representation of an optical vital signs sensor according to aspects of the invention. In this aspect of the invention, an alternative to the solution of FIG. 3 is depicted. Here, only a single light emitting diode 111 is provided while several photo diodes 121-12n at different positions are provided. The light emitting diode 111 is arranged between the photo diodes 121-12n. Optionally, the positions of the photo diodes 121-12n and the light emitting device 111 are arranged in a line. As mentioned in the previous aspect of the invention, the control unit 140 may control the operation of the light emitting diode 111 as well as the photo diodes 121-12n to achieve an output signal with a good signal quality while still taking care of the power consumption of the sensor.

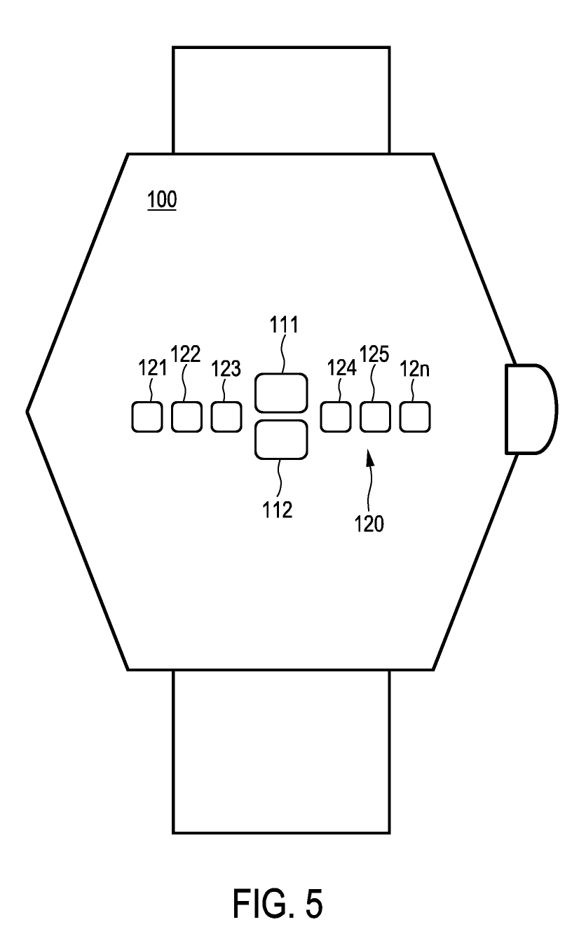

FIG. 5 shows a basic representation of an optical vital signs sensor according to aspects of the invention. According to this aspect of the invention, two light emitting diodes 111, 112 are provided which are arranged next to or adjacent to each other. The arrangement of the photo diodes 121-2n corresponds to the aspect of the invention according to FIG. 4. The operation of the control unit 140 corresponds to the operation of the control unit 140 according to FIG. 2.

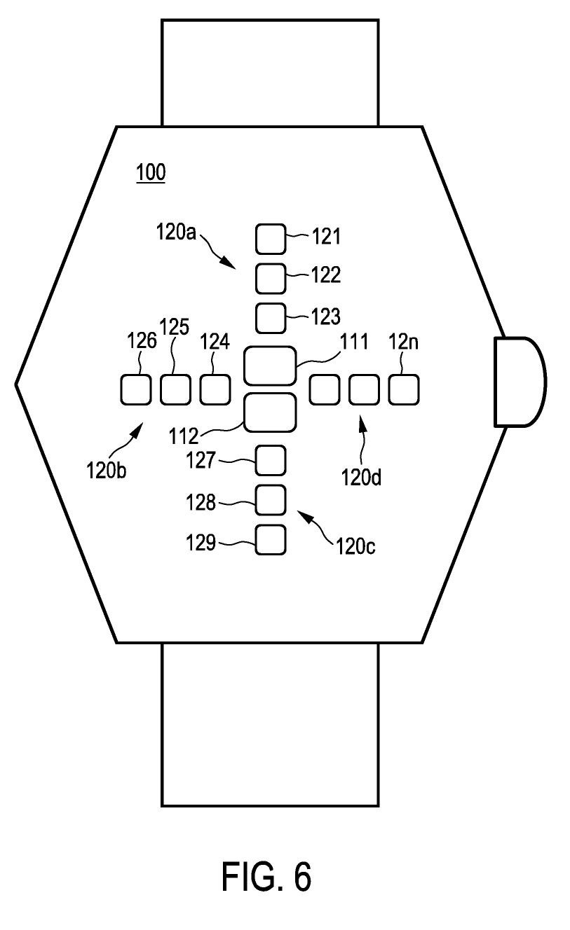

FIG. 6 shows a basic representation of an optical vital signs sensor according to aspects of the invention. The optical vital signs sensor 100 according to the aspect of the invention of FIG. 6 substantially corresponds to the optical vital signs sensor according to FIG. 5 but the photo detector 120 comprises four arms 120a, 120b, 120c, 120d each of several photo diodes, wherein the four arms are arranged in form of a cross, with the first and second light emitting diode 111, 112 being arranged at the centre of the cross. The operation of the control unit 140 according to this aspect corresponds to the operation of the control unit 140 according to the aspect of FIG. 2.

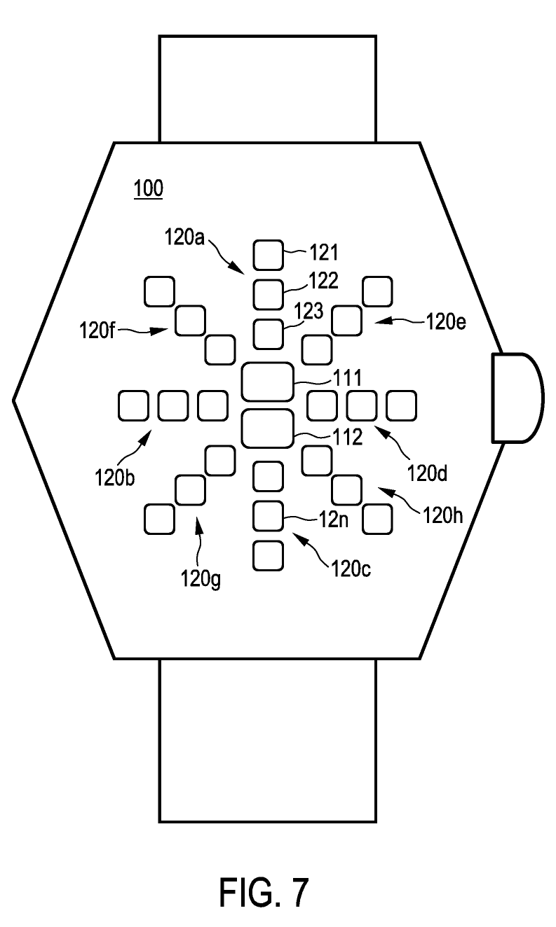

FIG. 7 shows a basic representation of an optical vital signs sensor according to aspects of the invention. The optical vital signs sensor 100 according to this aspect of the invention is based on the optical vital signs sensor according to FIG. 6. However, the photo detector 120 comprises six arms 120a-120h each having several photo diodes, wherein the arms 120a-120h are arranged around the first and second light emitting diode 111, 112 which are arranged in the centre of the arms.

The operation of the control unit according to the aspect of FIG. 7 corresponds to the operation of the control unit 140 according to the aspect of FIG. 2.

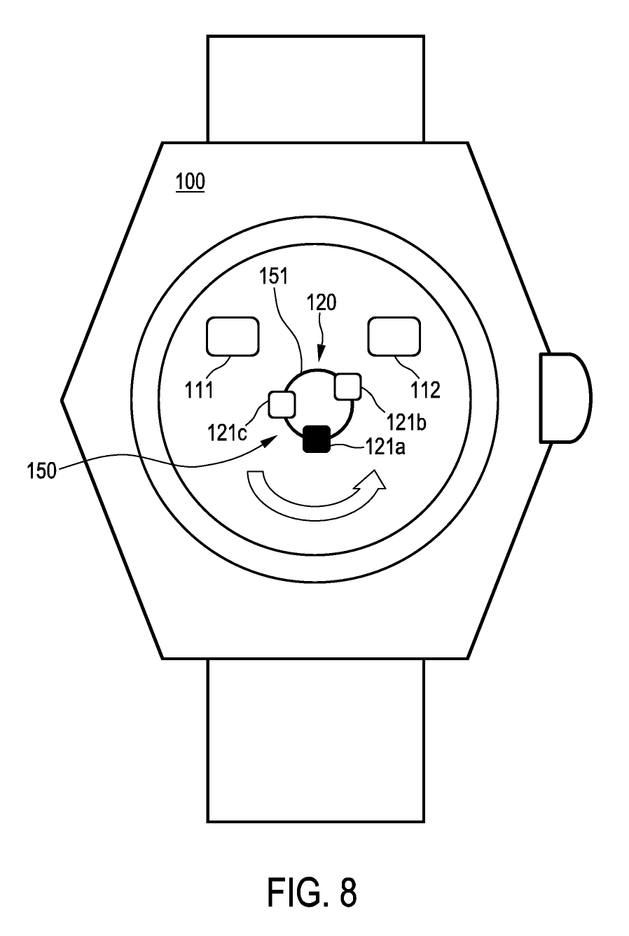

FIG. 8 shows a representation of an optical vital signs sensor according to aspects of the invention. The optical vital signs sensor 100 according to this aspect of the invention comprises a light source with at least one light emitting diode 111, 112 as well as a photo detector with at least one photo diode 121. The photo detector 120 with the at least one photo diode 121 can be coupled to an adjusting unit 150 which can adjust the distance between the at least one light emitting diode 111, 112 and the at least one photo diode 121. The adjusting unit 150 is preferably a mechanical adjusting unit. Optionally, the adjusting unit 150 comprises an adjusting ring 151 which is coupled to the at least one photo diode 121. By turning the ring 151 either electrically or mechanically, the at least one photo diode 121 is rotated. In FIG. 8, three different positions 121a, 121b, 121c of the photo diode 121 are depicted. It should be noted that more positions are possible. Thus, by turning the ring 151, the distance between the at least one light emitting diode 111, 112 and the at least one photo diode 121 can be adjusted. The signal analysis unit 141 analyses the output signal of the photo detector 120 during the activation of the adjusting unit to determine a position of the at least one photo diode 121 at which the best signal quality for example in turns of signal-to-noise ratio, AC or DC amplitude or the ratio between AC and DC of the output signal is achieved.

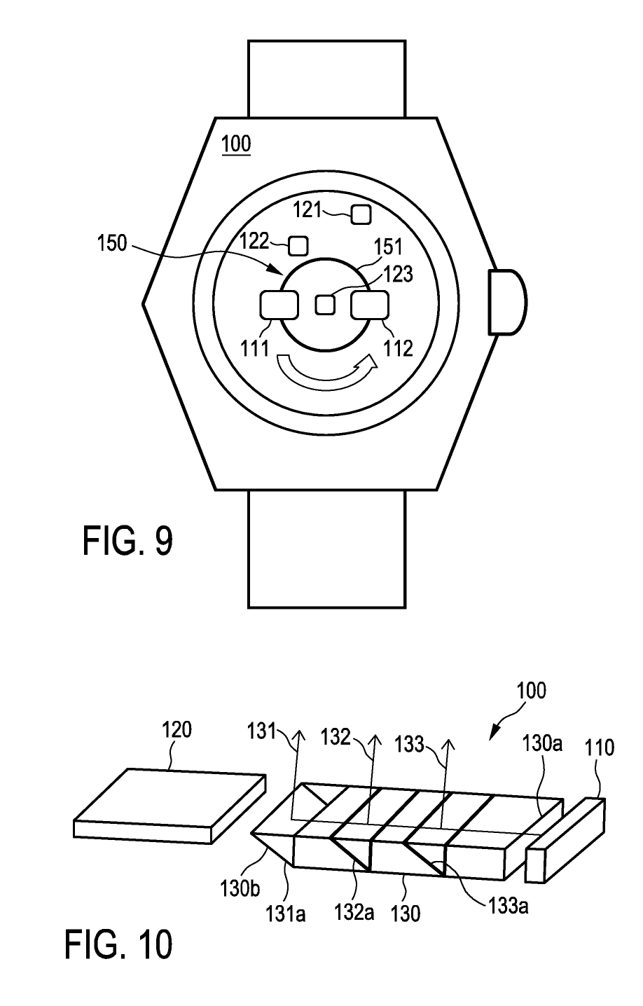

FIG. 9 shows a representation of an optical vital signs sensor according to aspects of the invention. According to this aspect of the invention, the optical vital signs sensor 100 comprises three different photo diodes 121-23 and at least one light emitting diode 111, 112 which are arranged on a ring 151 of an adjustment unit 150. With the adjusting unit 150, the position of the at least one light emitting diode 111, 112 can be adjusted relative to the positions of the at least one photo diode 121, 123. Thus, the distance between the photo diode and the light emitting diodes can be adjusted.

This can either be performed mechanically by a user which can directly or indirectly turn the ring 151. In addition or alternatively, the control unit 140 may control the turning of the ring in the aspect according to FIG. 8 as well as in the aspect according to FIG. 9. According to this aspect of the invention, the signal analysis unit 141 can analyse the output signal of the photo detector during the activation of the adjusting unit 150, i.e. the turning of the ring 151. If the optimal signal quality is achieved, the adjusting by means of the adjusting unit 50 can be stopped.

According to an aspect of the invention, the optical vital signs sensor according to FIG. 8 or FIG. 9 can be combined with an optical vital signs sensor according to one of the FIGS. 2 to 7. Thus, the mechanical adjustment by means of the adjustment unit and the adjustment ring 151 can be combined with the arrangement of the light emitting diode 111, 112 and the photo diodes 121-12n of FIG. 3, FIG. 4, FIG. 5, FIG. 6 or FIG. 7.

By means of the mechanical adjustment of the light emitting diodes and the photo diodes, the length of the optical path between at least one light emitting diode and at least one photo diode can be adjusted. This can be performed for example according to the skin type or morphology of the user.

According to one aspect of the invention, only one pair of light emitting diodes and one pair of photo sensors can be used. Thus, a low complex and low cost arrangement can be achieved while still being able to adjust the length of the optical light path according to the skin type and morphology of the user.

According to an aspect of the invention, the arrangement of the light emitting diodes 111, 112 as well as the photo detectors 121-2n according to the embodiments of FIGS. 8 and 9 can optionally correspond to the arrangements of the light emitting diodes and photo diodes according to the FIG. 3, 4, 5, 6 or 7.

According to an aspect of the invention, the adjustment of the length of the optical light path in an optical vital signs sensor according to the invention is performed by mechanically adjusting the relative or absolute positions of the light emitting diodes and the photo diodes.

FIG. 10 shows a schematic representation of an optical vital signs sensor according to an aspect of the invention. The optical vital signs sensor 100 according to this aspect of the invention comprises a light source 110 for example in form of a light emitting diode as well as a photo detector in form of a photo diode 120. Between the light emitting diode 110 and the photo detector 120, a light guide 130 can be arranged. The light guide 130 comprises a first end 130a and a second end 130b. The first end 130a is coupled or is directed towards the light source 110, i.e. light from the light source 110 is coupled into the light guide 130. The light guide 130 comprises several for example voltage dependent reflective elements 131-133 which can be activated by a supply voltage or control voltage.

The voltage dependent reflective elements 131-133 are arranged at different positions. During operation, the control unit 140 can activate one of the voltage dependent reflective elements 131-133 such that the light from the light source 110 travels through the light guide 130 and exits the light guide at one of the voltage dependent reflective elements 131-133. As the voltage dependent reflective elements 131-133 are arranged at different positions, by controlling the voltage dependent reflective elements, a position where the light enters the skin 1000 of the user can be adjusted or the distance between the light leaving the light guide and the photo detector 120 can be adjusted. As in the other aspects of the invention, the signal analysis unit 141 can detect the output signal of the photo detector 120 while several of the voltage dependent reflective elements 131-133 are activated in order to determine that voltage dependent reflective elements at which the signal quality of the photo detector is at its best.

According to the aspects of the invention, the control unit 140 can control the effective distance between the active light source and the active photo detector or photo diode.

According to a further aspect of the invention, a mechanical adjusting unit may also be introduced according to the aspect of FIG. 10. Here, in addition to the operation of the light guide 130, a mechanical adjusting unit may be provided to further mechanically adjust the position of the light guide 130.

Other variations of the disclosed embodiment can be understood and effected by those skilled in the art in practicing the claimed invention from a study of the drawings, the disclosure and the appended claims.

In the claims, the word “comprising” does not exclude other elements or steps and in the indefinite article “a” or “an” does not exclude a plurality.

A single unit or device may fulfill the functions of several items recited in the claims. The mere fact that certain measures are recited in mutual different dependent claims does not indicate that a combination of these measurements cannot be used to advantage. A computer program may be stored/distributed on a suitable medium such as an optical storage medium or a solid state medium, supplied together with or as a part of other hardware, but may also be distributed in other forms such as via the internet or other wired or wireless telecommunication systems.

Any reference signs in the claims should not be construed as limiting the scope.

1. Optical vital signs sensor, comprising:
a light source having at least one light unit each configured to generate light, wherein the light of the at least one light unit is directed towards the skin of a user,
at least one photo detector unit having at least one photo diode configured to detect light which is indicative of a reflection of the light from the at least one light unit in or from the skin of the user, and
an adjusting unit configured to adjust an effective distance which the light travels between the at least one light unit and the at least one photo diode wherein the adjusting unit is configured to mechanically adjust a position where the light from the at least one light unit enters the skin of the user or to mechanically adjust a position of one of the at least one light unit relative to a position of one of the at least one photo diode thereby adjusting a distance which the light travels between an active light unit and an active photo diode,
wherein the adjusting unit comprises an adjusting ring which is coupled to the at least one photo diode or to the at least one light unit.
wherein by turning the ring the at least one photo diode or the at least one light unit can be adjusted relative to the position of the at least one light unit or the at least one photo diode,
wherein the adjusting ring is directly or indirectly mechanically turnable by a user.

2. Optical vital signs sensor according to claim 1, wherein the at least one photo detector unit comprises a plurality of photo diodes configured to detect light which is indicative of a reflection of the light from the at least one light unit in or from the skin of the user, and further comprising a control unit configured to control an operation of the light source and/or the photo detector unit based on a signal quality of an output signal of the photo detector analyzed by a signal analyzing unit in the control unit by selecting and activating at least one light unit from among the at least one light unit and/or at least one photo diode from among the plurality of photo diodes thereby further adjusting a distance which the light travels between an active light unit and an active photo diode or further adjusting a position where the light from the at least one light source enters the skin of a user.

3. Optical vital signs sensor according to claim 2, wherein the signal quality is analyzed by the signal analyzing unit regarding at least one of a signal-to-noise ratio, an amplitude of the DC component of the output signal, an amplitude of the AC component of the output signal and a ratio between the AC and the DC component of the output signal of the photo detector.

4. Optical vital signs sensor according to claim 1, further comprising a control unit configured to control an adjustment operation of the adjusting unit based on a signal quality of an output signal of the photo detector analyzed by a signal analyzing unit in the control unit thereby adjusting a distance which the light travels between an active light unit and an active photo diode.

5. Optical vital signs sensor according to claim 4, wherein the signal quality is analyzed by the signal analyzing unit regarding at least one of a signal-to-noise ratio, an amplitude of the DC component of the output signal, an amplitude of the AC component of the output signal and a ratio between the AC and the DC component of the output signal of the photo detector.

6. Optical vital signs sensor according to claim 1, further comprising a light guide having a plurality of controllable reflective units being arranged adjacent to the at least one light unit and being configured to direct the light from the at least one light unit towards the skin of the user when activated thereby adjusting the distance the light travels between the at least one light unit and the at least one photo diode.

7. A method of operating an optical vital signs sensor which comprises a light source having at least one light unit and at least one photo detector having at least one photo diode comprising the steps of:
directing the light of the light source towards a skin of a user,
detecting light which is indicative of a reflection of the light from the at least one light unit in or from the skin of a user by the at least one photo diode, and
mechanically adjusting a position where the light enters the skin of the user, and/or
mechanically adjusting a distance between the at least one light unit and the at least one photo diode,
turning an adjusting ring which is coupled to the at least one photo diode or to the at least one light unit,
wherein by turning the ring the at least one photo diode or the at least one light unit can be adjusted relative to the position of the at least one light unit or the at least one photo diode,
wherein the adjusting ring is directly or indirectly mechanically turnable by a user.

8. A computer program product comprising a computer readable memory storing computer program code means for causing the optical vital signs sensor to carry out the steps of the method of operating an optical vital signs sensor according to claim 7.
